(19) United States
(12) Patent Application Publication
(10) Pub. No.: US 2026/0167781 A1
(43) Pub. Date: Jun. 18, 2026

(54) SILICONE HYDROGELS FORMED FROM MONOFUNCTIONAL UREA-BASED SILICONE MONOMERS

(71) Applicant: Bausch + Lomb Ireland Limited, Dublin (IE)

(72) Inventors: Jamie Homnick, Webster, NY (US); Yongxing Qiu, Rochester, NY (US)

(21) Appl. No.: 19/421,417

(22) Filed: Dec. 16, 2025

Related U.S. Application Data
(60) Provisional application No. 63/734,897, filed on Dec. 17, 2024.

(51) Int. Cl. C08J 3/075; A61F 2/16; C08J 3/24; C08K 5/101; C08K 5/20

(52) U.S. Cl. CPC C08J 3/075; A61F 2/16; C08J 3/24; C08K 5/101; C08K 5/20; C08J 2383/08; C08J 2439/06; C08J 2477/00

(57) ABSTRACT

An ophthalmic device which is a polymerization product of a monomeric mixture including (a) one or more monofunctional urea-based silicone monomers represented by a structure of Formula I:

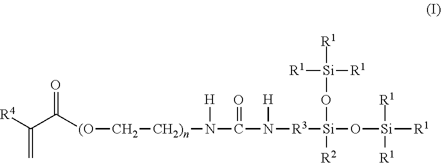

wherein each R¹ is independently an alkyl group; R² is an alkyl group or a trialkyl siloxy group, R³ is an alkylene group, R⁴ is hydrogen or methyl and n is an integer from 1 to 12, (b) one or more ophthalmic device-forming hydrophilic comonomers or polymers, and (c) one or more cross-linking agents. The ophthalmic device is optically clear.

### PRIORITY CLAIM

The present application claims priority to U.S. Provisional Patent Application Ser. No. 63/734,897, entitled “Silicone Hydrogels Formed Monofunctional Urea-Based Silicone Monomers,” filed Dec. 17, 2024, the content of which is incorporated by reference herein in its entirety.

### BACKGROUND

In the field of biomedical devices such as contact lenses, various physical and chemical properties such as, for example, optical clarity, oxygen permeability, wettability, material strength and stability are but a few of the factors that must be carefully balanced in order to provide a useable contact lens. For example, since the cornea receives its oxygen supply exclusively from contact with the atmosphere, good oxygen permeability is a critical characteristic for any contact lens material. Wettability also is important in that, if the lens is not sufficiently wettable, it does not remain lubricated and therefore cannot be worn comfortably in the eye. Accordingly, the optimum contact lens would have at least both excellent oxygen permeability and excellent tear fluid wettability.

Hydrogels represent a desirable class of materials for many biomedical applications, including contact lenses and intraocular lenses. Hydrogels are hydrated, crosslinked polymeric systems that contain water in an equilibrium state. Silicone hydrogels are a known class of hydrogels and are characterized by the inclusion of a silicone-containing material. Typically, a silicone-containing monomer is copolymerized by free radical polymerization with a hydrophilic monomer, with either the silicone-containing monomer or the hydrophilic monomer functioning as a crosslinking agent (a crosslinker being defined as a monomer having multiple polymerizable functionalities) or a separate crosslinker may be employed. An advantage of silicone hydrogels over non-silicone hydrogels is that the silicone hydrogels typically have higher oxygen permeability due to the inclusion of the silicone-containing monomer.

### SUMMARY

In accordance with an aspect of the present disclosure, an ophthalmic device which is a polymerization product of a monomeric mixture comprising:
(a) one or more monofunctional urea-based silicone monomers represented by a structure of Formula I:

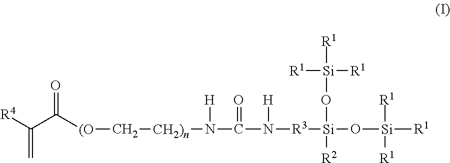

wherein R¹, R², R³, R⁴ and n are as defined herein,
(b) one or more ophthalmic device-forming hydrophilic comonomers or polymers, and
(c) one or more cross-linking agents,
wherein the ophthalmic device is optically clear.

In accordance with another aspect of the present disclosure, a method for making an ophthalmic device comprises:
(a) curing in a mold a polymerization product of a monomeric mixture comprising:
(i) one or more monofunctional urea-based silicone monomers represented by a structure of Formula I:

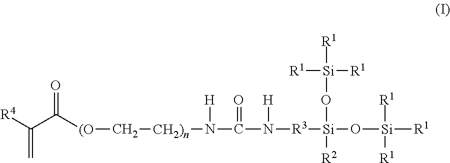

wherein R¹, R², R³, R⁴ and n are as defined herein,
(ii) one or more ophthalmic device-forming hydrophilic comonomers or polymers, and
(iii) one or more cross-linking agents, and
(b) dry releasing the polymerization product from the mold,
wherein the ophthalmic device is optically clear.

### DETAILED DESCRIPTION

Various illustrative embodiments described herein are directed to monofunctional urea-based silicone monomers and their use in forming ophthalmic devices such as silicon hydrogels having improved optical clarity.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

As used herein, the term “SiHy” shall be understood to mean silicone hydrogel.

As used herein, the term “hydrogel” or “hydrogel material” refers to a crosslinked polymeric material that has three-dimensional polymer networks (i.e., polymer matrix), is insoluble in water, but can hold at least 10 percent by weight of water in its polymer matrix when it is fully hydrated.

As used herein, the term “silicone hydrogel” or “SiHy” interchangeably refers to a hydrogel containing silicone. A silicone hydrogel (SiHy) typically is obtained by copolymerization of a polymerizable composition comprising at least one silicone-containing vinylic monomer or at least one silicone-containing vinylic macromer or at least one silicone-containing prepolymer having ethylenically unsaturated groups.

As used herein, the term “ophthalmic device” refers to ophthalmic devices that reside in or on the eye. These devices can provide optical correction, wound care, drug delivery, diagnostic functionality or cosmetic enhancement or effect or a combination of these properties. Suitable ophthalmic devices include, for example, ophthalmic lenses such as soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking.

As used herein, the term “(meth)” denotes an optional methyl substituent. Thus, terms such as “(meth)acrylate” denotes either methacrylate or acrylate, and “(meth)acrylamide” denotes either methacrylamide or acrylamide.

As used in this disclosure, the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

The terms “wt. %,” “vol. %” or “mol. %” refers to a weight, volume, or molar percentage of a component, respectively, based on the total weight, the total volume, or the total moles of material that includes the component. In a non-limiting example, 10 moles of component in 100 moles of the material are 10 mol. % of component.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

Silicone hydrogels (SiHy) such as contact lenses, which are made of a hydrated, crosslinked polymeric material that contains silicone and a certain amount of water within the lens polymer matrix at equilibrium, are increasingly becoming popular, because they have minimal adverse effects on corneal health due to their high oxygen permeability. However, incorporation of silicone in a contact lens material can have undesirable effects on the hydrophilicity and wettability of silicone hydrogels, because silicone is hydrophobic and has a great tendency to migrate onto the lens surface being exposed to air. Contact lens manufacturers have therefore made a great effort in developing SiHy contact lenses having a hydrophilic and wettable surface that are optically clear.

One approach to modifying the hydrophilicity and wettability of a SiHy contact lens is the incorporation of monomeric wetting/comfort agents such as non-functionalized comfort polymers, e.g., high molecular weight hydrophilic polymer(s) (e.g., polyvinylpyrrolidone (PVP)) in the monomeric mixtures for forming interpenetrating networks in a lens formulation for making naturally-wettable SiHy contact lens (i.e., wettable SiHy lenses without post-molding surface treatment). However, not all silicone containing monomers display compatibility with the high molecular weight hydrophilic polymers. For example, while it may be possible to incorporate the high molecular weight hydrophilic polymers as internal wetting/comfort agents into silicone hydrogel lenses, such polymers can be difficult to obtain an optically clear lens after autoclaving.

The illustrative embodiments described herein overcome the foregoing drawbacks by using one or more of the monofunctional urea-based silicone monomers represented by the structure of Formula I to form an ophthalmic device such as a SiHy contact lens having improved properties such as hydrophilicity and wettability and is optically clear. The one or more monofunctional urea-based silicone monomers represented by the structure of Formula I are compatible with the ophthalmic device-forming hydrophilic comonomers or polymers in the monomeric mixtures to form optically clear contact lenses.

In accordance with one or more non-limiting illustrative embodiments, an ophthalmic device as disclosed herein is a polymerization product of a monomeric mixture comprising:
(a) one or more monofunctional urea-based silicone monomers represented by a structure of Formula I:

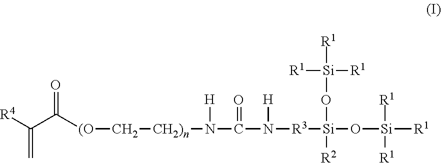

wherein each R¹ is independently an alkyl group; R² is an alkyl group or a trialkyl siloxy group, R³ is an alkylene group, R⁴ is hydrogen or methyl and n is an integer from 1 to 12, and
(b) one or more ophthalmic device-forming hydrophilic comonomers or polymers,
wherein the ophthalmic device is optically clear.

Representative examples of alkyl groups for use herein include, by way of example, a straight or branched alkyl chain radical containing carbon and hydrogen atoms of from 1 to about 30 carbon atoms, or alternatively from 1 to about 12 carbon atoms, or alternatively from 1 to about 6 carbon atoms, or alternatively from 1 to about 3 carbon atoms with or without unsaturation, to the rest of the molecule, e.g., methyl, ethyl, n-propyl, 1-methylethyl (isopropyl), n-butyl, n-pentyl, etc., and the like.

Representative examples of alkylene groups for use herein include, by way of example, a straight or branched alkyl chain radical containing carbon and hydrogen atoms of from 1 to about 12 carbon atoms, or alternatively from 1 to about 6 carbon atoms, or alternatively from 1 to about 3 carbon atoms with or without unsaturation, to the rest of the molecule, e.g., methylene, ethylene, propylene, butylene, etc., and the like.

In one embodiment, R¹ and R² are independently a C₁ to C₆ alkyl group; R³ is a C₁ to C₆ alkylene group; and n is from 1 to 6.

In one embodiment, R¹ and R² are independently a C₁ to C₃ alkyl group; R³ is a C₁ to C₃ alkylene group; and n is from 1 to 3.

In one embodiment, R¹ and R² are independently a C₁ to C₃ alkyl group; R³ is a C₁ to C₃ alkylene group; and n is from 3 to 5.

In one embodiment, R¹ is a C₁ to C₆ alkyl group; R² is a tri C₁ to C₆ alkyl siloxy group, R³ is a C₁ to C₆ alkylene group; and n is from 1 to 6.

In one embodiment, R¹ is a C₁ to C₃ alkyl group; R² is a tri C₁ to C₃ alkyl siloxy group, R³ is a C₁ to C₃ alkylene group; and n is from 1 to 3.

In one embodiment, R¹ is a C₁ to C₃ alkyl group; R² is a tri C₁ to C₃ alkyl siloxy group, R³ is a C₁ to C₃ alkylene group; and n is from 3 to 5.

In an illustrative embodiment, the monofunctional urea-based silicone monomer represented by the structure of Formula I disclosed herein can be prepared based on the known reaction between an isocyanate group and an amino group to form a urea linkage. For example, an isocyanate-containing acrylate (or methacrylate) can react with an aminoalkyl-tris(trialkylsiloxy) silane or an aminoalkyl-bis(trialkylsiloxy)alkyl silane to form a vinylic monomer of Formula I.

Suitable isocyanatoalkylacrylates and isocyanatoalkylacrylates include, for example, isocyanatomethylacrylate, isocyanatoethylacrylate, isocyanatopropylacrylate, isocyanatoisopropylacrylate, isocyanatobutylacrylate, isocyanatopentylacrylate, isocyanatohexylylacrylate, isocyanatoheptylacrylate, isocyanatomethylmethacrylate, isocyanatoethylmethacrylate, isocyanatopropylmethacrylate, isocyanatoisopropylmethacrylate, isocyanatobutylmethacrylate, isocyanatopentylmethacrylate, isocyanatohexylmethacrylate, and isocyanatoheptylmethacrylate. Suitable aminoalkyl-tris(trimethylsiloxy) silanes include, for example, aminoethyl-tris(trimethylsiloxy) silane, aminopropyl-tris(trimethylsiloxy) silane, aminobutyl-tris(trimethylsiloxy) silane, aminopentyl-tris(trimethylsiloxy) silane, aminohexyl-tris(trimethylsiloxy) silane, and aminoheptyl-tris(trimethylsiloxy) silane. Suitable aminoalkyl-bis(trialkylsiloxy)alkyl silanes include, for example, aminoethylmethyl-bis(trimethylsiloxy) silane, aminopropylmethyl-bis(trimethylsiloxy) silane, aminobutylmethyl-bis(trimethylsiloxy) silane, aminopentylmethyl-bis(trimethylsiloxy) silane, aminohexylmethyl-bis(trimethylsiloxy) silane, and aminoheptylmethyl-bis(trimethylsiloxy) silane.

The monomeric mixture can include the one or more monofunctional urea-based silicone monomers represented by a structure of Formula I in an amount ranging from about 10 wt. % to about 45 wt. %, based on the total weight of the monomeric mixture. In some embodiments, the monomeric mixture can include the one or more monofunctional urea-based silicone monomers represented by a structure of Formula I in an amount ranging from about 10 wt. % to about 30 wt. %, based on the total weight of the monomeric mixture.

The monomeric mixture further includes ophthalmic device-forming hydrophilic comonomers or polymers as component (b). Suitable ophthalmic device-forming hydrophilic comonomers or polymers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include, but are not limited to, methacrylic acid, acrylic acid and the like and mixtures thereof. Representative examples of acrylamides include, but are not limited to, alkylamides such as N,N-dimethylacrylamide, N,N-dimethylmethacrylamide and the like and mixtures thereof. Representative examples of cyclic lactams include, but are not limited to, N-vinyl-2-pyrrolidone, N-vinyl caprolactam, N-vinyl-2-piperidone and the like and mixtures thereof. Representative examples of hydroxyl-containing (meth)acrylates include, but are not limited to, 2-hydroxyethyl methacrylate (HEMA), 2-hydroxyethyl acrylate (HEA), glycerol methacrylate and the like and mixtures thereof. Additional ophthalmic device-forming hydrophilic comonomers or polymers include, for example, the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable ophthalmic device-forming hydrophilic comonomers or polymers will be apparent to one skilled in the art. Mixtures of the foregoing ophthalmic device-forming hydrophilic comonomers or polymers can also be used in the monomeric mixtures herein.

In some embodiments, the ophthalmic device-forming hydrophilic comonomers as component (b) can be one or more hydrophilic acrylate comonomers. Suitable one or more hydrophilic acrylate comonomers include, for example, alkylamides such as N,N-dimethylacrylamide, N,N-dimethylmethacrylamide and the like, hydroxyl-containing acrylates such as 2-hydroxyethyl acrylate (HEA) and the like and mixtures thereof.

The monomeric mixture can include the one or more ophthalmic device-forming hydrophilic comonomers or polymers in an amount ranging from about 10 wt. % to about 90 wt. %, based on the total weight of the monomeric mixture. In some embodiments, the monomeric mixture can include the one or more ophthalmic device-forming hydrophilic comonomers or polymers in an amount ranging from about 10 wt. % to about 50 wt. %, based on the total weight of the monomeric mixture.

In non-limiting illustrative embodiments, as may be combined with one or more of the preceding paragraphs, the monomeric mixture further includes one or more crosslinking agents. Suitable crosslinking agents for use herein are known in the art. For example, in non-limiting illustrative embodiments, suitable one or more cross-linking agents include one or more crosslinking agents containing at least two ethylenically unsaturated reactive end groups. In one embodiment, the ethylenically unsaturated reactive end groups are (meth)acrylate-containing reactive end groups. In another embodiment, the ethylenically unsaturated reactive end groups are non-(meth)acrylate reactive end groups. In one embodiment, the ethylenically unsaturated reactive end groups are a combination of one or more (meth)acrylate-containing reactive end groups and one or more non-(meth)acrylate reactive end groups.

In an illustrative embodiment, useful one or more crosslinking agents containing at least two ethylenically unsaturated reactive end groups include, for example, one or more di-, tri- or tetra(meth)acrylate-containing crosslinking agents. In an illustrative embodiment, useful one or more di-, tri- or tetra(meth)acrylate-containing crosslinking agents include, for example, alkanepolyol di-, tri- or tetra(meth)acrylate-containing crosslinking agents such as, for example, one or more alkylene glycol di(meth)acrylate crosslinking agents, one or more alkylene glycol tri(meth)acrylate crosslinking agents, one or more alkylene glycol tetra(meth)acrylate crosslinking agents, one or more alkanediol di(meth)acrylate crosslinking agents, alkanediol tri(meth)acrylate crosslinking agents, alkanediol tetra(meth)acrylate crosslinking agents, agents, one or more alkanetriol di(meth)acrylate crosslinking agents, alkanetriol tri(meth)acrylate crosslinking agents, alkanetriol tetra(meth)acrylate crosslinking agents, agents, one or more alkanetetraol di(meth)acrylate crosslinking agents, alkanetetraol tri(meth)acrylate crosslinking agents, alkanetetraol tetra(meth)acrylate crosslinking agents and the like and mixtures thereof.

In an illustrative embodiment, one or more alkylene glycol di(meth)acrylate crosslinking agents include tetraethylene glycol dimethacrylate, ethylene glycol di(meth)acrylates having up to about 10 ethylene glycol repeating units, butyleneglycol di(meth)acrylate and the like. In one embodiment, one or more alkanediol di(meth)acrylate crosslinking agents include butanediol di(meth)acrylate crosslinking agents, hexanediol di(meth)acrylate and the like. In one embodiment, one or more alkanetriol tri(meth)acrylate crosslinking agents are trimethylol propane trimethacrylate crosslinking agents. In one embodiment, one or more alkanetetraol tetra(meth)acrylate crosslinking agents are pentaerythritol tetramethacrylate crosslinking agents.

In a non-limiting illustrative embodiment, suitable crosslinking agents include, for example, ethylene glycol diacrylate, diethylene glycol diacrylate, allyl acrylate, 1,3-propanediol diacrylate, 2,3-propanediol diacrylate, 1,6-hexanediol diacrylate, 1,4-butanediol diacrylate, triethylene glycol diacrylate, cyclohexane-1,1-diyldimethanol diacrylate, 1,4-cyclohexanediol diacrylate, 1,3-adamantanediol diacrylate, 1,3-adamantanedimethyl diacrylate, 2,2-diethyl-1,3-propanediol diacrylate, 2,2-diisobutyl-1,3-propanediol diacrylate, 1,3-cyclohexanedimethyl diacrylate, 1,4-cyclohexanedimethyl diacrylate; neopentyl glycol diacrylate, tetraethyleneglycol diacrylate, polyethyleneglycol diacrylate; and their corresponding methacrylates.

In a non-limiting illustrative embodiment, suitable crosslinking agents include, for example, 1,4-butanediol diacrylate, 1,4-butanediol dimethacrylate, ethylene glycol dimethacrylate, diethylene glycol diacrylate, diethylene glycol dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate (Mn=700 Daltons), poly(ethylene glycol) dimethacrylate (Mn=700 Daltons), and poly(ethylene glycol) dimethacrylate (Mn=1000 Daltons).

In one embodiment, the one or more crosslinking agents containing at least two ethylenically unsaturated reactive end groups include at least one allyl-containing reactive end group and at least one (meth)acrylate-containing reactive end group. In an illustrative embodiment, the one or more crosslinking agents can be allyl methacrylate.

The monomeric mixture can include the one or more crosslinking agents in an amount ranging from about 0.1 wt. % to about 10 wt. %, based on the total weight of the monomeric mixture.

In non-limiting illustrative embodiments, as may be combined with one or more of the preceding paragraphs, the monomeric mixture can further include one or more polyamides. As used herein, the term “polyamide” refers to polymers and copolymers comprising repeating units containing amide groups. In some embodiments, the polyamide may comprise cyclic amide groups and may be any polyamide known to those of skill in the art. A cyclic polyamide for use herein comprises cyclic amide groups and are capable of association with, for example, hydroxyl groups.

Suitable cyclic amides that can be used to form the cyclic polyamides include, for example, α-lactam, β-lactam, γ-lactam, δ-lactam, and ε-lactam. A representative example of a suitable cyclic polyamide includes cyclic polyamide polymers and cyclic polyamide copolymers comprising repeating units of Formula II:

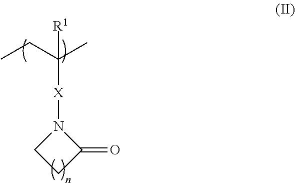

wherein R¹ is a hydrogen atom or methyl group; n is a number from 1 to 10; X is a direct bond, —(CO)—, or —(CONHR²—)—, wherein R² is a C₁ to C₃ alkyl group.

Suitable cyclic polyamides for use in the monomeric mixture disclosed herein includes a cyclic polyamide having a weight average molecular weight of at most 300,000 Daltons (Da), or alternatively a cyclic polyamide having a weight average molecular weight of at most about 100,000 Da, or alternatively a cyclic polyamide having at most about a weight average molecular weight of at most about 90,000 Da, or alternatively a cyclic polyamide having at most about a weight average molecular weight of at most about 80,000 Da, or alternatively a cyclic polyamide having at most about a weight average molecular weight of at most about 70,000 Da, or alternatively a cyclic polyamide having at most about a weight average molecular weight of at most about 60,000 Da. At the same time, a cyclic polyamide for use in the monomeric mixture disclosed herein includes a cyclic polyamide having a weight average molecular weight of at least about 500 Da, or alternatively a cyclic polyamide having a weight average molecular weight of at least about 1,000 Da, or alternatively a cyclic polyamide having a weight average molecular weight of at least about 2,000 Da, or alternatively a cyclic polyamide having a weight average molecular weight of at least about 3,000 Da. Alternatively, a cyclic polyamide for use in the monomeric mixture disclosed herein includes a cyclic polyamide having a weight average molecular weight of from about 500 Da to 300,000 Da, or alternatively a cyclic polyamide having a weight average molecular weight of from about 1,000 Da to 100,000 Da, or alternatively a cyclic polyamide having a weight average molecular weight of from about 2,000 Da to about 90,000 Da, or alternatively a cyclic polyamide having a weight average molecular weight of from about 3,000 Da to about 80,000 Da, or alternatively a cyclic polyamide having a weight average molecular weight of from about 3,000 Da to about 60,000 Da. The weight average molecular weight of the cyclic polyamide can be determined by gel permeation chromatography (GPC).

When X is a direct bond and n may be 2, the cyclic polyamide may be a polyvinylpyrrolidone (PVP). Suitable polyvinylpyrrolidones for use in the monomeric mixture disclosed herein includes a polyvinylpyrrolidone having a weight average molecular weight of at most 300,000 Daltons (Da), or alternatively a polyvinylpyrrolidone having a weight average molecular weight of at most about 100,000 Da, or alternatively a polyvinylpyrrolidone having at most about a weight average molecular weight of at most about 90,000 Da, or alternatively a polyvinylpyrrolidone having at most about a weight average molecular weight of at most about 80,000 Da, or alternatively a polyvinylpyrrolidone having at most about a weight average molecular weight of at most about 70,000 Da, or alternatively a polyvinylpyrrolidone having at most about a weight average molecular weight of at most about 60,000 Da. At the same time, a polyvinylpyrrolidone for use in the monomeric mixture disclosed herein includes a polyvinylpyrrolidone having a weight average molecular weight of at least about 500 Da, or alternatively a polyvinylpyrrolidone having a weight average molecular weight of at least about 1,000 Da, or alternatively a polyvinylpyrrolidone having a weight average molecular weight of at least about 2,000 Da, or alternatively a polyvinylpyrrolidone having a weight average molecular weight of at least about 3,000 Da. Alternatively, a polyvinylpyrrolidone for use in the monomeric mixture disclosed herein includes a polyvinylpyrrolidone having a weight average molecular weight of from about 500 Da to 300,000 Da, or alternatively a polyvinylpyrrolidone having a weight average molecular weight of from about 1,000 Da to 100,000 Da, or alternatively a polyvinylpyrrolidone having a weight average molecular weight of from about 2,000 Da to about 90,000 Da, or alternatively a polyvinylpyrrolidone having a weight average molecular weight of from about 3,000 Da to about 80,000 Da.

The cyclic polyamides may comprise 50 mole percent or more, or at least 70 mole percent, or at least 80 mole percent of the repeating unit of Formula II.

The monomeric mixture can include the one or more cyclic polyamides in an amount ranging from about 0.1 wt. % to about 10 wt. %, based on the total weight of the monomeric mixture. In some embodiments, the monomeric mixture can include the one or more cyclic polyamides in an amount ranging from about 0.5 wt. % to about 2 wt. %, based on the total weight of the monomeric mixture.

In non-limiting illustrative embodiments, as may be combined with one or more of the preceding paragraphs, the monomeric mixture can further include one or more functionalized comfort polymers. In non-limiting illustrative embodiments, the one or more functionalized comfort polymers include, for example, a functionalized poloxamer, a functionalized poloxamine and mixtures thereof. A functionalized poloxamer is derived from a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly(ethylene oxide)-poly(propylene oxide)-poly(ethylene oxide) blocks as generally represented by the structure:

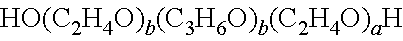

wherein a is independently at least 1 and b is at least 1.

Reverse Pluronics are a series of BAB block copolymers, respectively composed of poly(propylene oxide)-poly(ethylene oxide)-poly(propylene oxide) blocks as generally represented by the structure:

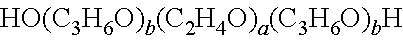

wherein a is at least 1 and b is independently at least 1. The poly(ethylene oxide), PEO, blocks are hydrophilic, whereas the poly(propylene oxide), PPO, blocks are hydrophobic in nature. The poloxamers in each series have varying ratios of PEO and PPO which ultimately determines the hydrophilic-lipophilic balance (HLB) of the material, i.e., the varying HLB values are based upon the varying values of a and b, a representing the number of hydrophilic poly(ethylene oxide) units (PEO) being present in the molecule and b representing the number of hydrophobic poly(propylene oxide) units (PPO) being present in the molecule. In one embodiment, the poloxamer will have an HLB ranging from about 5 to about 24. In another embodiment, the poloxamer will have an HLB ranging from about 1 to about 5.

Poloxamers and reverse poloxamers have terminal hydroxyl groups that can be terminal functionalized to form the functionalized poloxamer. An example of a terminal functionalized poloxamer as discussed herein is poloxamer dimethacrylate (e.g., Pluronic® F127 dimethacrylate) as disclosed in U.S. Patent Application Publication No. 2003/0044468 and U.S. Pat. No. 9,309,357, the contents of which are incorporated by reference herein. Other examples include glycidyl-terminated copolymers of polyethylene glycol and polypropylene glycol as disclosed in U.S. Pat. No. 6,517,933, the contents of which are incorporated by reference herein.

The poloxamer is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomeric mixture. The term block copolymer as used herein shall be understood to mean a poloxamer as having two or more blocks in their polymeric backbone(s). In non-limiting illustrative embodiments, a functionalized poloxamer is a poloxamer di(meth)acrylate, a reverse poloxamer di(meth)acrylate and mixtures thereof.

While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure:

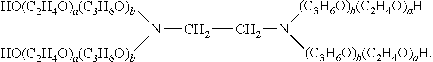

wherein a is independently at least 1 and b is independently at least 1.

The poloxamine can be functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended ophthalmic device forming monomeric mixture. The term block copolymer as used herein shall be understood to mean a poloxamine as having two or more blocks in their polymeric backbone(s).

The monomeric mixture can include the one or more functionalized comfort polymers in an amount ranging from about 1 wt. % to about 10 wt. %, based on the total weight of the monomeric mixture. In some embodiments, the monomeric mixture can include the one or more functionalized comfort polymers in an amount ranging from about 2 wt. % to about 7 wt. %, based on the total weight of the monomeric mixture.

In non-limiting illustrative embodiments, as may be combined with one or more of the preceding paragraphs, the monomeric mixture can further include a reactive (polymerizable) ultraviolet (UV) light absorber and/or a reactive blue-light absorber. Suitable reactive UV light absorbers can be any known reactive UV absorber. In non-limiting illustrative embodiments, suitable reactive UV light absorbers include, for example, 2-(2′-hydroxy-3′-methallyl-5′-methylphenyl)benzotriazole, commercially available as o-Methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl) phenoxy)ethyl methacrylate.

In one illustrative embodiment, suitable UV light absorbers include, for example, one or more compounds of the following formulae:

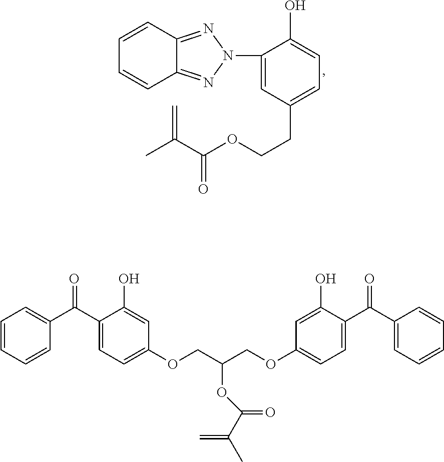 [SMILES: C=C(C)C(=O)OCCc1ccc(O)c(-n2nc3ccccc3n2)c1 ; C=C(C)C(=O)OC(COc1ccc(C(=O)c2ccccc2)c(O)c1)COc1ccc(C(=O)c2ccccc2)c(O)c1]

### (2-Propenoic acid, 2-methyl,2-(4-benzoyl-3-hydroxyphenoxy)-1-[(4-benzoyl3-hydroxyphenoxy)methyl ester)

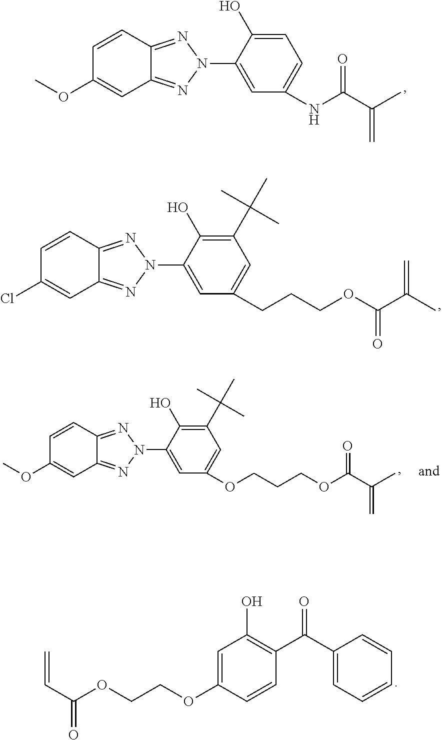

These compounds are merely illustrative and not intended to be limiting. Any known UV blocker or later developed UV blocker are contemplated for use herein.

The monomeric mixture can include the UV light absorbers in an amount ranging from about 0.1 wt. % to about 5 wt. %, based on the total weight of the monomeric mixture. In another illustrative embodiment, the monomeric mixture can include the UV light absorbers in an amount ranging from about 1.5 wt. % to about 2.5 wt. %, based on the total weight of the monomeric mixture.

Many reactive blue-light absorbing compounds are known. Preferred reactive blue-light absorbing compounds are those described in U.S. Pat. Nos. 5,470,932; 8,207,244; and 8,329,775, the contents of which are hereby incorporated by reference. In one embodiment, a blue-light absorbing dye is N-2-[3-(2′-methylphenylazo)-4-hydroxyphenyl]ethyl methacrylamide. In some embodiments, the monomeric mixture can include the blue-light absorbers in an amount ranging from about 0.005 wt. % to about 1 wt. %, based on the total weight of the monomeric mixture. In some embodiments, the monomeric mixture can include the blue-light absorbers in an amount ranging from about 0.01 wt. % to about 1 wt. %, based on the total weight of the monomeric mixture.

The monomeric mixture may further contain, as necessary and within limits not to impair the purpose and effect of the illustrative embodiments, various additives such as an antioxidant, a coloring agent, a lubricant, an internal wetting agent, a toughening agent and the like and other constituents as are well known in the art.

In non-limiting illustrative embodiments, as may be combined with one or more of the preceding paragraphs, the ophthalmic devices disclosed herein can be a high-water content silicone ophthalmic device such as silicone hydrogel having an equilibrium water content of at least about 35 wt. %. In another illustrative embodiment, the high-water content silicone ophthalmic device disclosed herein can have an equilibrium water content of at least about 50 wt. %. In another illustrative embodiment, the high-water content silicone ophthalmic device disclosed herein can have an equilibrium water content of at least about 60 wt. %. In another illustrative embodiment, the high-water content silicone ophthalmic device disclosed herein can have an equilibrium water content of at least about 70 wt. %. In another illustrative embodiment, the ophthalmic devices disclosed herein can be a high-water content silicone ophthalmic device having an equilibrium water content of from about 35 wt. % to about 80 wt. %.

The ophthalmic devices of the illustrative embodiments, e.g., contact lenses or intraocular lenses, can be prepared by polymerizing the foregoing monomeric mixtures to form a product that can be subsequently formed into the appropriate shape by, for example, lathing, injection molding, compression molding, cutting and the like. For example, in producing contact lenses, the initial mixture may be polymerized in tubes to provide rod-shaped articles, which are then cut into buttons. The buttons may then be lathed into contact lenses.

Alternately, the ophthalmic devices such as contact lenses may be cast directly in molds, e.g., polypropylene molds, from the mixtures, e.g., by spincasting and static casting methods. Spincasting methods are disclosed in U.S. Pat. Nos. 3,408,429 and 3,660,545, and static casting methods are disclosed in U.S. Pat. Nos. 4,113,224, 4,197,266, and 5,271,875. Spincasting methods involve charging the mixtures to be polymerized to a mold, and spinning the mold in a controlled manner while exposing the mixture to a radiation source such as UV light. Static casting methods involve charging the monomeric mixture between two mold sections, one mold section shaped to form the anterior lens surface and the other mold section shaped to form the posterior lens surface, and curing the mixture while retained in the mold assembly to form a lens, for example, by free radical polymerization of the mixture. Examples of free radical reaction techniques to cure the lens material include thermal radiation, infrared radiation, electron beam radiation, gamma radiation, ultraviolet (UV) radiation, and the like; or combinations of such techniques may be used. U.S. Pat. No. 5,271,875 describes a static cast molding method that permits molding of a finished lens in a mold cavity defined by a posterior mold and an anterior mold. As an additional method, U.S. Pat. No. 4,555,732 discloses a process where an excess of a monomeric mixture is cured by spincasting in a mold to form a shaped article having an anterior lens surface and a relatively large thickness, and the posterior surface of the cured spincast article is subsequently lathed to provide a contact lens having the desired thickness and posterior lens surface.

Polymerization may be facilitated by exposing the mixture to heat (thermal cure) and/or radiation, such as ultraviolet light, visible light, or high energy radiation. A polymerization initiator may be included in the mixture to facilitate the polymerization step. Representative examples of free radical thermal polymerization initiators include organic peroxides such as acetyl peroxide, lauroyl peroxide, decanoyl peroxide, stearoyl peroxide, benzoyl peroxide, tertiarylbutyl peroxypivalate, peroxydicarbonate, and the like. Representative examples of diazo initiators include VAZO 64, and VAZO 67. Representative UV initiators are those known in the art and include benzoin methyl ether, benzoin ethyl ether, Darocure® 1173, 1164, 2273, 1116, 2959, 3331 (EM Industries) and Irgacure® 651 and 184 (Ciba-Geigy). Representative visible light initiators include IRGACURE 819 and other phosphine oxide-type initiators, and the like. Generally, the initiator will be employed in the monomeric mixture at a concentration of about 0.01 to about 5 wt. % of the total mixture.

Polymerization is generally performed in a reaction medium, such as, for example, a solution or dispersion using a diluent or solvent, e.g., water, methoxypropyl acetate or an alkanol containing from 1 to 4 carbon atoms such as methanol, ethanol or propan-2-ol. Alternatively, a mixture of any of the above solvents may be used.

Generally, polymerization can be carried out for about 15 minutes to about 72 hours, and under an inert atmosphere of, for example, nitrogen or argon. If desired, the resulting polymerization product can be dried under vacuum, e.g., for about 5 to about 72 hours, or left in an aqueous solution prior to use.

Polymerization of the mixtures will yield a polymer, that when hydrated, preferably forms a hydrogel. When producing a hydrogel lens, the mixture may further include at least a diluent as discussed above that is ultimately replaced with water when the polymerization product is hydrated to form a hydrogel. Generally, the water content of the hydrogel is as described hereinabove, i.e., at least about 50 wt. %. The amount of diluent used should be less than about 50 wt. % and in most cases, the diluent content will be less than about 30 wt. %. However, in a particular polymer system, the actual limit will be dictated by the solubility of the various monomers in the diluent. In order to produce an optically clear copolymer, it is important that a phase separation leading to visual opacity does not occur between the comonomers and the diluent, or the diluent and the final copolymer.

Furthermore, the maximum amount of diluent which may be used will depend on the amount of swelling the diluent causes the final polymers. Excessive swelling will or may cause the copolymer to collapse when the diluent is replaced with water upon hydration. Suitable diluents include, but are not limited to, ethylene glycol; glycerine; liquid poly(ethylene glycol); alcohols; alcohol/water mixtures; ethylene oxide/propylene oxide block copolymers; low molecular weight linear poly(2-hydroxyethyl methacrylate); glycol esters of lactic acid; formamides; ketones; dialkylsulfoxides; butyl carbitol; borates as discussed herein and the like and mixtures thereof.

If necessary, it may be desirable to remove residual diluent from the lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include wetting agents known in the prior art for making hydrogel materials.

In the case of intraocular lenses, the monomeric mixtures to be polymerized may further include a monomer for increasing the refractive index of the resultant polymerized product. Examples of such monomers include aromatic (meth)acrylates, such as phenyl (meth)acrylate, 2-phenylethyl (meth)acrylate, 2-phenoxyethyl methacrylate, and benzyl (meth)acrylate.

The ophthalmic devices such as contact lenses obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The lens may then be turned over in order to machine the other side of the lens.

The lens may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The following examples are provided to enable one skilled in the art to practice the invention and are merely illustrative. The examples should not be read as limiting the scope of the invention as defined in the claims.

In the examples, the following abbreviations are used.
IEM: 2-Isocyanatoethyl methacrylate.
IEEM: 2-(2-Isocyanatoethoxy)ethyl methacrylate.
Tris-NH₂: 3-Aminopropyl-tris(trimethylsiloxy) silane.
Bis-NH₂: 3-Aminopropylmethyl-bis(trimethylsiloxy) silane.
MPA: Methoxypropyl acetate.
HEA: 2-Hydroxyethyl acrylate.
HEMA: 2-Hydroxyethyl methacrylate.
TRIS MA: Tris (trimethoxysilylpropyl)methacrylate.
NVP: N-vinyl-2-pyrrolidone.
DMA: N,N-dimethylacrylamide.
EGDMA: Ethylene glycol dimethacrylate.
MPC: Methacryloyl phosphorylcholine.
Irg819: Irgacure 819 photoinitiator.
PVP-1: Polyvinylpyrrolidone having a weight average molecular weight of 1,300,000 Da as determined by gel permeation chromatography (GPC).
PVP-2: Polyvinylpyrrolidone having a weight average molecular weight of 360,000 Da as determined by GPC.
PVP-3: Polyvinylpyrrolidone having a weight average molecular weight of 55,000 Da as determined by GPC.
PVP-4: Polyvinylpyrrolidone having a weight average molecular weight of 29,000 Da as determined by GPC.
PVP-5: Polyvinylpyrrolidone having a weight average molecular weight of 8,000 Da as determined by GPC.
PVP-6: Polyvinylpyrrolidone having a weight average molecular weight of 3,500 Da as determined by GPC.

### Example 1

Preparation of a monofunctional urea-based silicone monomer (Tris-IEM) having the following structure:

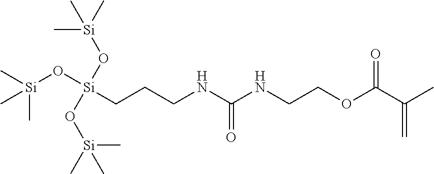 [SMILES: C=C(C)C(=O)OCCNC(=O)NCCC[Si](O[Si](C)(C)C)(O[Si](C)(C)C)O[Si](C)(C)C]

by the general reaction scheme.

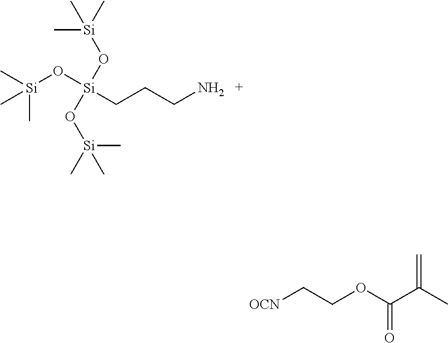

A glass jar was fitted with a magnetic stir bar and a thermocouple and clamped onto a magnetic stirring plate. The glass jar was charged with 4.388 g (28.28 mmol) IEM, and 10.005 g (28.28 mmol) Tris-NH₂ was added dropwise keeping exotherm under 60° C. After the Tris-NH₂ addition was complete, the reaction mixture was allowed to cool gradually to room temperature yielding a viscous but pourable clear, colorless liquid in quantitative yield. H-NMR in CDCl₃ confirmed the compound in high purity.

### Example 2

Preparation of a monofunctional urea-based silicone monomer (Tris-IEEM) having the following structure:

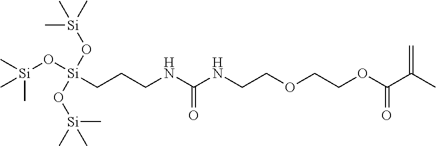 [SMILES: C=C(C)C(=O)OCCOCCNC(=O)NCCC[Si](O[Si](C)(C)C)(O[Si](C)(C)C)O[Si](C)(C)C]

by the general reaction scheme.

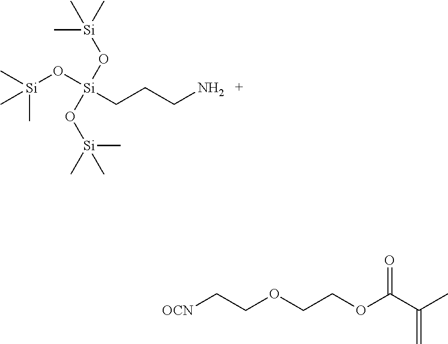

A glass jar was fitted with a magnetic stir bar and a thermocouple and clamped onto a magnetic stirring plate. The glass jar was charged with 5.635 g (28.28 mmol) IEEM, and 10.004 g (28.28 mmol) Tris-NH₂ was added dropwise keeping exotherm under 60° C. After the Tris-NH₂ addition was complete, the reaction mixture was allowed to cool gradually to room temperature yielding a viscous but pourable clear, colorless liquid in quantitative yield. H-NMR in CDCl₃ confirmed the compound in high purity.

### Example 3

Preparation of a monofunctional urea-based silicone monomer (Bis-IEM) having the following structure:

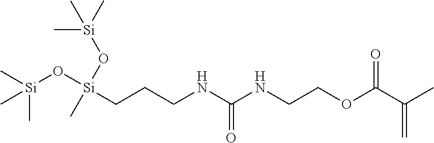 [SMILES: C=C(C)C(=O)OCCNC(=O)NCCC[Si](C)(O[Si](C)(C)C)O[Si](C)(C)C]

by the general reaction scheme.

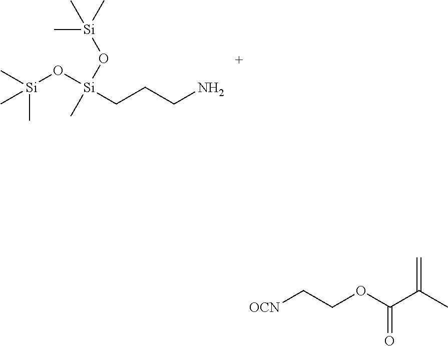

A glass jar was fitted with a magnetic stir bar and a thermocouple and clamped onto a magnetic stirring plate. The glass jar was charged with 5.550 g (35.77 mmol) IEM, and 10.001 g (35.77 mmol) Bis-NH₂ was added dropwise keeping exotherm under 60° C. After the Bis-NH₂ addition was complete, the reaction mixture was allowed to cool gradually to room temperature yielding a viscous but pourable clear, colorless liquid in quantitative yield. H-NMR in CDCl₃ confirmed the compound in high purity.

### Example 4

Preparation of a monofunctional urea-based silicone monomer (Bis-IEEM) having the following structure:

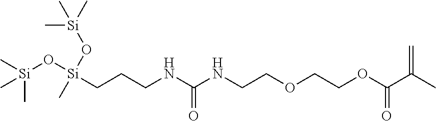 [SMILES: C=C(C)C(=O)OCCOCCNC(=O)NCCC[Si](C)(O[Si](C)(C)C)O[Si](C)(C)C]

by the general reaction scheme.

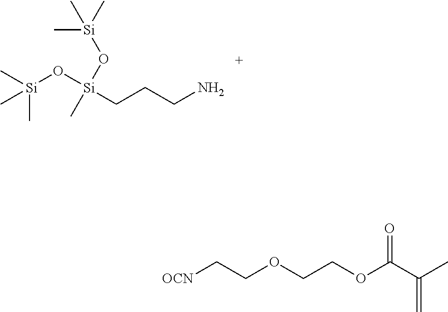

A glass jar was fitted with a magnetic stir bar and a thermocouple and clamped onto a magnetic stirring plate. The glass jar was charged with 7.132 g (35.80 mmol) IEEM, and 10.005 g (35.78 mmol) Bis-NH₂ was added dropwise keeping exotherm under 55° C. After the Bis-NH₂ addition was complete, the reaction mixture was allowed to cool gradually to room temperature yielding a viscous but pourable clear, colorless liquid in quantitative yield. This material eventually solidified into a waxy solid when stored at 4° C. and remained solid when warmed to room temperature. H-NMR in CDCl₃ confirmed the compound in high purity.

### Example 5

An ophthalmic device was made from a polymerizable composition of a 1:1 weight ratio mixture of Tris-IEM:HEA.

Tris-IEM (0.5 g) of Example 1 was combined with HEA (0.5 g) and 0.01 g Irg819. A few drops were aliquoted into a lens mold and irradiated for 20 seconds with an LED curing light centered at 450 nm (blue light, ˜3 W/cm²). The mixture polymerized into a clear, colorless solid.

### Example 6

An ophthalmic device was made from a polymerizable composition of a 1:1 weight ratio mixture of Tris-IEEM:HEA.

Tris-IEEM (0.5 g) of Example 2 was combined with HEA (0.5 g) and 0.01 g Irg819. A few drops were aliquoted into a lens mold and irradiated for 20 seconds with an LED curing light centered at 450 nm (blue light, ˜3 W/cm²). The mixture polymerized into a clear, colorless solid.

### Example 7

An ophthalmic device was made from a polymerizable composition of a 1:1 weight ratio mixture of Bis-IEM:HEA.

Bis-IEM (0.5 g) of Example 3 was combined with HEA (0.5 g) and 0.01 g Irg819. A few drops were aliquoted into a lens mold and irradiated for 20 seconds with an LED curing light centered at 450 nm (blue light, ˜3 W/cm²). The mixture polymerized into a clear, colorless solid.

### Example 8

An ophthalmic device was made from a polymerizable composition of a 1:1 weight ratio mixture of Bis-IEEM:HEA.

Bis-IEEM (0.5 g) of Example 4 was combined with HEA (0.5 g) and 0.01 g Irg819. A few drops were aliquoted into a lens mold and irradiated for 20 seconds with an LED curing light centered at 450 nm (blue light, ˜3 W/cm²). The mixture polymerized into a clear, colorless solid.

### Comparative Example A

Tris-MA (0.5 g) was combined with HEMA (0.5 g) and 0.01 g Irg819. A few drops were aliquoted into a lens mold and irradiated for 20 seconds with an LED curing light centered at 450 nm (blue light, ˜3 W/cm²). No solid polymer was apparent by visual observation.

### Example 9

A monomeric mix was made by mixing the following components, listed in Table 1 at amounts per weight.

TABLE 1

| Formulation | Wt. % |
|---|---|
| Tris-IEM of Ex. 1 | 45.5 |
| 16.78% MPC:HEA | 45.1 |
| IRG819 | 1.1 |
| EGDMA | 2.1 |
| MPA diluent | 6.3 |

The 16.78% MPC:HEA mixture was made by mixing MPC and HEA, listed in Table 2 at amounts per weight.

TABLE 2

| Formulation | Wt. % |
|---|---|
| MPC | 16.78 |
| HEA | 83.22 |

The resultant monomeric mixture was cast into contact lenses as follows. Irg819 was dissolved in 16.78% MPC:HEA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were clear and colorless.

### Comparative Example B

A monomeric mix was made by mixing the following components, listed in Table 3 at amounts per weight.

TABLE 3

| Formulation | Wt. % |
|---|---|
| Tris-IEM of Ex. 1 | 45.3 |
| 2.84% PVP-1:HEA | 22.9 |
| 1.23% PVP-1:DMA | 22.8 |
| IRG819 | 0.7 |
| EGDMA | 1.2 |
| MPA diluent | 7.1 |

The 2.84% PVP-1:HEA mixture was made by mixing PVP-1 and HEA, listed in Table 4 at amounts per weight.

TABLE 4

| Formulation | Wt. % |
|---|---|
| PVP-1 | 2.84 |
| HEA | 97.16 |

The 1.23% PVP-1:DMA mixture was made by mixing PVP-1 and DMA, listed in Table 5 at amounts per weight.

TABLE 5

| Formulation | Wt. % |
|---|---|
| PVP-1 | 1.23 |
| DMA | 98.77 |

The resultant monomeric mixture was cast into contact lenses as follows. Irg819 was dissolved in 2.84% PVP-1:HEA and 1.23% PVP-1:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in a borated buffered solution (BBS), sealed in glass vials, and autoclaved. The dry released contact lenses were clear and colorless, and the autoclaved lenses were cloudy.

### Comparative Example C

A monomeric mix was made by mixing the following components, listed in Table 6 at amounts per weight.

TABLE 6

| Formulation | Wt. % |
|---|---|
| Tris-IEM of Ex. 1 | 45.7 |
| 2.84% PVP-1:6.30% MPC:HEA | 22.8 |
| 1.23% PVP-1:DMA | 22.5 |
| IRG819 | 1.1 |
| EGDMA | 1.2 |
| MPA diluent | 7.0 |

The 2.84% PVP-1:6.30% MPC:HEA mixture was made by mixing PVP-1, MPC and HEA, listed in Table 7 at amounts per weight.

TABLE 7

| Formulation | Wt. % |
|---|---|
| PVP-1 | 1.12 |
| MPC | 5.98 |
| HEA | 91.32 |

The resultant monomeric mixture was cast into contact lenses as follows. Irg819 was dissolved in 2.84% PVP-1:6.30% MPC:HEA and 1.23% PVP-1:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were cloudy.

### Comparative Example D

A monomeric mix was made by mixing the following components, listed in Table 8 at amounts per weight.

TABLE 8

| Formulation | Wt. % |
|---|---|
| Tris-IEM of Ex. 1 | 45.8 |
| 15% MPC:5.47% PVP-1:HEA | 45.8 |
| IRG819 | 0.5 |
| EGDMA | 1.0 |
| MPA diluent | 6.9 |

The 15% MPC:5.47% PVP-1:HEA mixture was made by mixing MPC, PVP and HEA, listed in Table 9 at amounts per weight.

TABLE 9

| Formulation | Wt. % |
|---|---|
| PVP-1 | 5.34 |
| MPC | 17.14 |
| HEA | 83.22 |

The resultant monomeric mixture was cast into contact lenses as follows. Irg819 was dissolved in 16.78% MPC:HEA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were cloudy.

### Comparative Example E

A monomeric mix was made by mixing the following components, listed in Table 10 at amounts per weight.

TABLE 10

| Formulation | Wt. % |
|---|---|
| Tris-IEM of Ex. 1 | 45.23 |
| 2.84% PVP-2:HEA | 22.86 |
| 1.23% PVP-2:DMA | 22.75 |
| IRG819 | 0.75 |
| EGDMA | 1.22 |
| MPA diluent | 7.20 |

The 2.84% PVP-2:HEA mixture was made by mixing PVP-2 and HEA, listed in Table 11 at amounts per weight.

TABLE 11

| Formulation | Wt. % |
|---|---|
| PVP-2 | 2.94 |
| HEA | 97.06 |

The 1.23% PVP-2:DMA mixture was made by mixing PVP-2 and DMA, listed in Table 12 at amounts per weight.

TABLE 12

| Formulation | Wt. % |
|---|---|
| PVP-2 | 1.19 |
| DMA | 98.81 |

The resultant monomeric mixture was cast into contact lenses as follows. Irg819 was dissolved in 2.84% PVP-2:HEA and 1.23% PVP-2:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were cloudy.

### Example 10

A monomeric mix was made by mixing the following components, listed in Table 13 at amounts per weight.

TABLE 13

| Formulation | Wt. % |
|---|---|
| Tris-IEM of Ex. 1 | 45.33 |
| 2.84% PVP-3:HEA | 22.92 |
| 1.23% PVP-3:DMA | 22.78 |
| IRG819 | 0.70 |
| EGDMA | 1.14 |
| MPA diluent | 7.13 |

The 2.84% PVP-3:HEA mixture was made by mixing PVP-3 and HEA, listed in Table 14 at amounts per weight.

TABLE 14

| Formulation | Wt. % |
|---|---|
| PVP-3 | 2.90 |
| HEA | 97.10 |

The 1.23% PVP-3:DMA mixture was made by mixing PVP-3 and DMA, listed in Table 15 at amounts per weight.

TABLE 15

| Formulation | Wt. % |
|---|---|
| PVP-3 | 1.29 |
| DMA | 98.71 |

The resultant monomeric mixture was cast into contact lenses as follows. Irg819 was dissolved in 2.84% PVP-3:HEA and 1.23% PVP-3:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were clear and colorless.

### Example 11

A monomeric mix was made by mixing the following components, listed in Table 16 at amounts per weight.

TABLE 16

| Formulation | Wt. % |
|---|---|
| Tris-IEM of Ex. 1 | 45.45 |
| 2.84% PVP-4:HEA | 22.86 |
| 1.23% PVP-4:DMA | 22.69 |
| IRG819 | 0.75 |
| EGDMA | 1.14 |
| MPA diluent | 7.12 |

The 2.84% PVP-4:HEA mixture was made by mixing PVP-4 and HEA, listed in Table 17 at amounts per weight.

TABLE 17

| Formulation | Wt. % |
|---|---|
| PVP-4 | 2.93 |
| HEA | 97.07 |

The 1.23% PVP-4:DMA mixture was made by mixing PVP-4 and DMA, listed in Table 18 at amounts per weight.

TABLE 18

| Formulation | Wt. % |
|---|---|
| PVP-4 | 1.17 |
| DMA | 98.83 |

The resultant monomeric mixture was cast into contact lenses as follows. Irg819 was dissolved in 2.84% PVP-4:HEA and 1.23% PVP-4:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were clear and colorless.

### Example 12

A monomeric mix was made by mixing the following components, listed in Table 19 at amounts per weight.

TABLE 19

| Formulation | Wt. % |
|---|---|
| Tris-IEM of Ex. 1 | 45.28 |
| 2.84% PVP-5:HEA | 22.76 |
| 1.23% PVP-5:DMA | 22.88 |
| IRG819 | 0.74 |
| EGDMA | 1.13 |
| MPA diluent | 7.21 |

The 2.84% PVP-5:HEA mixture was made by mixing PVP-5 and HEA, listed in Table 20 at amounts per weight.

TABLE 20

| Formulation | Wt. % |
|---|---|
| PVP-5 | 2.92 |
| HEA | 97.08 |

The 1.23% PVP-5:DMA mixture was made by mixing PVP-5 and DMA, listed in Table 21 at amounts per weight.

TABLE 21

| Formulation | Wt. % |
|---|---|
| PVP-5 | 1.22 |
| DMA | 98.78 |

The resultant monomeric mixture was cast into contact lenses as follows. Irg819 was dissolved in 2.84% PVP-5:HEA and 1.23% PVP-5:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were clear and colorless.

### Example 13

A monomeric mix was made by mixing the following components, listed in Table 22 at amounts per weight.

TABLE 22

| Formulation | Wt. % |
|---|---|
| Tris-IEM of Ex. 1 | 45.28 |
| 2.84% PVP-6:HEA | 23.05 |
| 1.23% PVP-6:DMA | 22.72 |
| IRG819 | 0.72 |
| EGDMA | 1.13 |
| MPA diluent | 7.10 |

The 2.84% PVP-6:HEA mixture was made by mixing PVP-6 and HEA, listed in Table 23 at amounts per weight.

TABLE 23

| Formulation | Wt. % |
|---|---|
| PVP-6 | 2.97 |
| HEA | 97.03 |

The 1.23% PVP-6:DMA mixture was made by mixing PVP-6 and DMA, listed in Table 24 at amounts per weight.

TABLE 24

| Formulation | Wt. % |
|---|---|
| PVP-6 | 1.23 |
| DMA | 98.77 |

The resultant monomeric mixture was cast into contact lenses as follows. Irg819 was dissolved in 2.84% PVP-6:HEA and 1.23% PVP-6:DMA. Next, EGDMA and MPA were added, followed by Tris-IEM. The mixture was stirred at room temperature. Contact lenses were formed by aliquoting 3 drops of the monomeric mixture into −3.00 Ultra molds and curing for 20 seconds each with an LED curing light centered at 450 nm (blue light, ˜3 W/cm²). The contact lenses were dry released, submerged in BBS, sealed in glass vials, and autoclaved. The dry released contact lenses were clear and colorless, and the autoclaved lenses were clear and colorless.

According to an aspect of the present disclosure, an ophthalmic device which is a polymerization product of a monomeric mixture comprises:
(a) one or more monofunctional urea-based silicone monomers represented by a structure of Formula I:

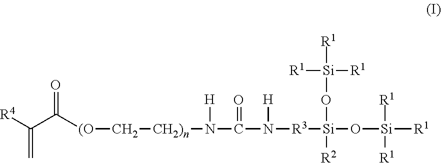

wherein each R¹ is independently an alkyl group; R² is an alkyl group or a trialkyl siloxy group, R³ is an alkylene group, R⁴ is hydrogen or methyl and n is an integer from 1 to 12,
(b) one or more ophthalmic device-forming hydrophilic comonomers or polymers, and
(c) one or more cross-linking agents,
wherein the ophthalmic device is optically clear.

In non-limiting illustrative embodiments, as may be combined with one or more of the preceding paragraphs, wherein in the one or more monofunctional urea-based silicone monomers, R¹ and R² are independently a C₁ to C₆ alkyl group; R³ is a C₁ to C₆ alkylene group; and n is from 1 to 6.

In non-limiting illustrative embodiments, as may be combined with one or more of the preceding paragraphs, wherein in the one or more monofunctional urea-based silicone monomers, R¹ and R² are independently a C₁ to C₃ alkyl group; R³ is a C₁ to C₃ alkylene group; and n is from 1 to 3.

In non-limiting illustrative embodiments, as may be combined with one or more of the preceding paragraphs, wherein in the one or more monofunctional urea-based silicone monomers, R¹ is a C₁ to C₆ alkyl group; R² is a tri C₁ to C₆ alkyl siloxy group, R³ is a C₁ to C₆ alkylene group; and n is from 1 to 6.

In non-limiting illustrative embodiments, as may be combined with one or more of the preceding paragraphs, wherein in the one or more monofunctional urea-based silicone monomers, R¹ is a C₁ to C₃ alkyl group; R² is a tri C₁ to C₃ alkyl siloxy group, R³ is a C₁ to C₃ alkylene group; and n is from 1 to 3.

In non-limiting illustrative embodiments, as may be combined with one or more of the preceding paragraphs, the one or more ophthalmic device-forming hydrophilic comonomers or polymers are selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycol) functionalized with polymerizable groups.

In non-limiting illustrative embodiments, as may be combined with one or more of the preceding paragraphs, the one or more ophthalmic device-forming hydrophilic comonomers are one or more of a hydrophilic (meth)acrylate comonomer and an acrylamide comonomer.

In non-limiting illustrative embodiments, as may be combined with one or more of the preceding paragraphs, the one or more ophthalmic device-forming hydrophilic comonomers are one or more of a 2-hydroxyethyl acrylate, 2-hydroxyethyl methacrylate and dimethylacrylamide.

In non-limiting illustrative embodiments, as may be combined with one or more of the preceding paragraphs, the monomeric mixture comprises:
about 10 wt. % to about 45 wt. %, based on the total weight of the monomeric mixture, of the monofunctional urea-based silicone monomers, and
about 10 wt. % to about 90 wt. %, based on the total weight of the monomeric mixture, of the one or more ophthalmic device-forming hydrophilic comonomers.

In non-limiting illustrative embodiments, as may be combined with one or more of the preceding paragraphs, the monomeric mixture further comprises a cyclic polyamide having a weight average molecular weight of at most 300,000 Daltons (Da).

In non-limiting illustrative embodiments, as may be combined with one or more of the preceding paragraphs, the monomeric mixture further comprises a polyvinylpyrrolidone having a weight average molecular weight of at most 300,000 Daltons (Da).

In non-limiting illustrative embodiments, as may be combined with one or more of the preceding paragraphs, the monomeric mixture further comprises a polyvinylpyrrolidone having a weight average molecular weight of at most 100,000 Da.

In non-limiting illustrative embodiments, as may be combined with one or more of the preceding paragraphs, the monomeric mixture further comprises a polyvinylpyrrolidone having a weight average molecular weight of at most 60,000 Da.

In non-limiting illustrative embodiments, as may be combined with one or more of the preceding paragraphs, the monomeric mixture comprises about 0.1 wt. % to about 10 wt. %, based on the total weight of the monomeric mixture, of the polyvinylpyrrolidone.

In non-limiting illustrative embodiments, as may be combined with one or more of the preceding paragraphs, the monomeric mixture comprises about 0.1 wt. % to about 10 wt. %, based on the total weight of the monomeric mixture, of a polyvinylpyrrolidone having a weight average molecular weight of at most 300,000 Da.

In non-limiting illustrative embodiments, as may be combined with one or more of the preceding paragraphs, the ophthalmic device is a silicone contact lens.

According to another aspect of the present disclosure, a method for making an ophthalmic device, comprises:
(a) curing in a mold a polymerization product of a monomeric mixture comprising:
(i) one or more monofunctional urea-based silicone monomers represented by a structure of Formula I:

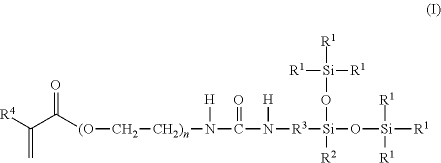

wherein each R¹ is independently an alkyl group; R² is an alkyl group or a trialkyl siloxy group, R³ is an alkylene group, R⁴ is hydrogen or methyl and n is an integer from 1 to 12,
(ii) one or more ophthalmic device-forming hydrophilic comonomers or polymers, and
(iii) one or more cross-linking agents, and
(b) dry releasing the polymerization product from the mold,
wherein the ophthalmic device is optically clear.

In non-limiting illustrative embodiments, as may be combined with one or more of the preceding paragraphs, wherein in the one or more monofunctional urea-based silicone monomers, R¹ is independently a C₁ to C₆ alkyl group; R² is a C₁ to C₆ alkyl group or a tri C₁ to C₆ alkyl siloxy group; R³ is a C₁ to C₆ alkylene group; and n is from 1 to 6.

In non-limiting illustrative embodiments, as may be combined with one or more of the preceding paragraphs, the monomeric mixture further comprises a polyvinylpyrrolidone having a weight average molecular weight of at most 300,000 Daltons (Da).

In non-limiting illustrative embodiments, as may be combined with one or more of the preceding paragraphs, the monomeric mixture comprises:
about 10 wt. % to about 45 wt. %, based on the total weight of the monomeric mixture, of the monofunctional urea-based silicone monomers;
about 10 wt. % to about 90 wt. %, based on the total weight of the monomeric mixture, of the one or more ophthalmic device-forming hydrophilic comonomers; and
about 0.1 wt. % to about 10 wt. %, based on the total weight of the monomeric mixture, of a polyvinylpyrrolidone having a weight average molecular weight of at most 300,000 Da.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. An ophthalmic device which is a polymerization product of a monomeric mixture comprising:
(a) one or more monofunctional urea-based silicone monomers represented by a structure of Formula I: 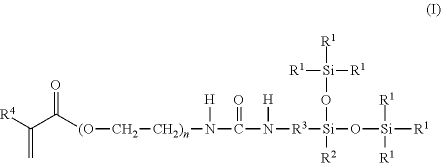
wherein each R¹ is independently an alkyl group; R² is an alkyl group or a trialkyl siloxy group, R³ is an alkylene group, R⁴ is hydrogen or methyl and n is an integer from 1 to 12;
(b) one or more ophthalmic device-forming hydrophilic comonomers or polymers; and
(c) one or more cross-linking agents;
wherein the ophthalmic device is optically clear.

2. The ophthalmic device according to claim 1, wherein in the one or more monofunctional urea-based silicone monomers, R¹ and R² are independently a C₁ to C₆ alkyl group; R³ is a C₁ to C₆ alkylene group; and n is from 1 to 6.

3. The ophthalmic device according to claim 1, wherein in the one or more monofunctional urea-based silicone monomers, R¹ and R² are independently a C₁ to C₃ alkyl group; R³ is a C₁ to C₃ alkylene group; and n is from 1 to 3.

4. The ophthalmic device according to claim 1, wherein in the one or more monofunctional urea-based silicone monomers, R¹ is a C₁ to C₆ alkyl group; R² is a tri C₁ to C₆ alkyl siloxy group, R³ is a C₁ to C₆ alkylene group; and n is from 1 to 6.

5. The ophthalmic device according to claim 1, wherein in the one or more monofunctional urea-based silicone monomers, R¹ is a C₁ to C₃ alkyl group; R² is a tri C₁ to C₃ alkyl siloxy group, R³ is a C₁ to C₃ alkylene group; and n is from 1 to 3.

6. The ophthalmic device according to claim 1, wherein the one or more ophthalmic device-forming hydrophilic comonomers or polymers are selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycol) functionalized with polymerizable groups.

7. The ophthalmic device according to claim 1, wherein the one or more ophthalmic device-forming hydrophilic comonomers are one or more of a hydrophilic (meth)acrylate comonomer and an acrylamide comonomer.

8. The ophthalmic device according to claim 1, wherein the one or more ophthalmic device-forming hydrophilic comonomers are one or more of a 2-hydroxyethyl acrylate, 2-hydroxyethyl methacrylate and dimethylacrylamide.

9. The ophthalmic device according to claim 1, wherein the monomeric mixture comprises:
about 10 wt. % to about 45 wt. %, based on the total weight of the monomeric mixture, of the monofunctional urea-based silicone monomers; and
about 10 wt. % to about 90 wt. %, based on the total weight of the monomeric mixture, of the one or more ophthalmic device-forming hydrophilic comonomers.

10. The ophthalmic device according to claim 1, wherein the monomeric mixture further comprises a cyclic polyamide having a weight average molecular weight of at most 300,000 Daltons (Da).

11. The ophthalmic device according to claim 1, wherein the monomeric mixture further comprises a polyvinylpyrrolidone having a weight average molecular weight of at most 300,000 Da.

12. The ophthalmic device according to claim 1, wherein the monomeric mixture further comprises a polyvinylpyrrolidone having a weight average molecular weight of at most 100,000 Da.

13. The ophthalmic device according to claim 1, wherein the monomeric mixture further comprises a polyvinylpyrrolidone having a weight average molecular weight of at most 60,000 Da.

14. The ophthalmic device according to claim 11, wherein the monomeric mixture comprises about 0.1 wt. % to about 10 wt. %, based on the total weight of the monomeric mixture, of the polyvinylpyrrolidone.

15. The ophthalmic device according to claim 9, wherein the monomeric mixture further comprises about 0.1 wt. % to about 10 wt. %, based on the total weight of the monomeric mixture, of a polyvinylpyrrolidone having a weight average molecular weight of at most 300,000 Da.

16. The ophthalmic device according to claim 1, which is a silicone contact lens.

17. A method for making an ophthalmic device, comprising:
(a) curing in a mold a polymerization product of a monomeric mixture comprising:
(i) one or more monofunctional urea-based silicone monomers represented by a structure of Formula I: 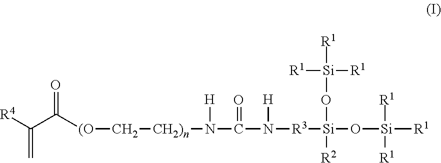
wherein each R¹ is independently an alkyl group; R² is an alkyl group or a trialkyl siloxy group, R³ is an alkylene group, R⁴ is hydrogen or methyl and n is an integer from 1 to 12;
(ii) one or more ophthalmic device-forming hydrophilic comonomers or polymers; and
(iii) one or more cross-linking agents; and
(b) dry releasing the polymerization product from the mold,
wherein the ophthalmic device is optically clear.

18. The method according to claim 17, wherein in the one or more monofunctional urea-based silicone monomers, R¹ is independently a C₁ to C₆ alkyl group; R² is a C₁ to C₆ alkyl group or a tri C₁ to C₆ alkyl siloxy group; R³ is a C₁ to C₆ alkylene group; and n is from 1 to 6.

19. The method according to claim 17, wherein the monomeric mixture further comprises a polyvinylpyrrolidone having a weight average molecular weight of at most 300,000 Daltons (Da).

20. The method according to claim 17, wherein the monomeric mixture comprises:
about 10 wt. % to about 45 wt. %, based on the total weight of the monomeric mixture, of the monofunctional urea-based silicone monomers;
about 10 wt. % to about 90 wt. %, based on the total weight of the monomeric mixture, of the one or more ophthalmic device-forming hydrophilic comonomers; and
about 0.1 wt. % to about 10 wt. %, based on the total weight of the monomeric mixture, of a cyclic polyamide having a weight average molecular weight of at most 300,000 Da.
